(19) United States
(12) Patent Application Publication
(10) Pub. No.: US 2002/0164335 A1
(43) Pub. Date: Nov. 7, 2002

(54) Compositions and methods for the study and diagnosis of prion diseases

(76) Inventors: David A. Harris, St. Louis, MO (US); Richard S. Stewart, St. Louis, MO (US)

Correspondence Address: Michael T. Marrah, Sonnenschein Nath & Rosenthal, Wacker Drive Station, Sears Tower, P.O. Box #061080, Chicago, IL, 60606-1080, US

(21) Appl. No.: 10/106,574

(22) Filed: Mar. 26, 2002

Related U.S. Application Data
(60) Provisional application No. 60/279,400, filed on Mar. 28, 2001.

(51) Int. Cl. A61K 39/395; G01N 33/53; C07K 16/40

(52) U.S. Cl. 424/146.1; 435/7.1; 530/388.26

(57) ABSTRACT

The present invention presents novel characteristics of a transmembrane form of PrP (^{Ctm}PrP) that is retained in the endoplasmic reticulum, and contains an uncleaved, N-terminal signal peptide as well as a C-terminal glycolipid anchor. The invention also identifies a mutant form of PrP that is synthesized exclusively with the ^{Ctm}PrP topology as well as related nucleic acid sequences and transfected cells and mammalian animals. Compositions and methods for the further study of ^{Ctm}PrP and mutant PrP, and their role in the causation and diagnosis of prion related diseases are further embodied in the invention.

### PRIORITY

This application claims the benefit of the earlier filed provisional application Serial No. 60/279,400.

### GOVERNMENT INTEREST

This invention was partly supported by grants from the National Institute of Health NINDS under at least Grant Nos. R01NS35496, T32NS07129 and F32NS41500, therefore, the government may have certain rights to the invention.

### FIELD OF THE INVENTION

This invention relates to the biochemical arts, including compositions and methods for the study and diagnosis of prion-caused diseases.

### BACKGROUND

Prion diseases are fatal neurodegenerative disorders characterized by dementia, ataxia, and cerebral spongiosis. A recent epidemic of bovine spongiform encephalopathy in the United Kingdom and the likely transmission of this disease to human beings has focussed public attention on the origin and transmission of prion disorders (Collinge, J., Variant Creutzfeldt-Jakob Disease, 354 LANCET 317-323 (1999), incorporated herein by reference). Infectious, inherited, and sporadic forms of these diseases are all due to conformational conversion of a normal cell-surface glycoprotein called PrP^{C}, expressed in neurons and glia, to a protease-resistant isoform denoted PrP^{Sc} (Harris, D. A., Cellular Biology of Prion Diseases, 12 CLIN. MICRO. REV. 429-444 (1999); Prusiner, S. B., Prion Biology and Diseases, COLD SPRING HARBOR LABORATORY PRESS, COLD SPRING HARBOR. 794 pp. (1999), both incorporated herein by reference). A great deal of evidence has accumulated indicating that PrP^{Sc} is infectious in the absence of nucleic acids, and that it is the principal component of infectious prion particles. It is also commonly assumed that PrP^{Sc} is the primary cause of neurodegeneration, based on the spatial and temporal correlation between the accumulation of this isoform and the degree of neuronal damage during the course of prion diseases (DeArmond, S. J., and Ironside, J. W., Neuropathology of prion Diseases, IN PRION BIOLOGY AND DISEASES, S. B. PRUSINER, EDITOR. COLD SPRING HARBOR LABORATORY PRESS, COLD SPRING HARBOR. 585-652 (1999), incorporated herein by reference).

Recently, however, an alternative topological variant of PrP called the C transmembrane form (“^{Ctm}PrP”) has been proposed as a key intermediate in infectious and inherited forms of prion disease. Whereas most molecules of PrP are anchored to the cell membrane exclusively by a C-terminal glycosyl-phosphatidylinositol (“GPI”) anchor (Lehmann, S., and Harris, D. A., A Mutant Prion Protein Displays an Aberrant Membrane Association When Expressed in Cultured Cells, 270 J. BIOL. CHEM. 24589-24597 (1995), incorporated herein by reference), ^{Ctm}PrP spans the membrane once via a conserved, hydrophobic segment encompassing residues 111-134, with the C-terminus on the exofacial surface (Hegde, R. S., Mastrianni, J. A., Scott, M. R., Defea, K. A., Tremblay, P., Torchia, M., DeArmond, S. J., Prusiner, S. B., and Lingappa, V. R., A Transmembrane Form of the Prion Protein in Neurodegenerative Disease, 279 SCIENCE 827-834 (1998a), incorporated herein by reference). A third topological variant of PrP, denoted N-transmembrane form (“^{Ntm}PrP”), spans the membrane via the same hydrophobic domain, but in the opposite orientation (N-terminus on the exofacial surface) (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE. 827-834 (1998a)).

Transmembrane forms of PrP were originally observed after in vitro translation of PrP mRNA on rabbit reticulocyte or wheat germ ribosomes in the presence of canine pancreatic microsomes (De Fea, K. A., Nakahara, D. H., Calayag, M. C., Yost, C. S., Mirels, L. F., Prusiner, S. B., and Lingappa, V. R., Determinants of Carboxyl-terminal Domain Translocation During Prion Protein Biogenesis, 269 J. BIOL. CHEM. 16810-16820 (1994); Hay, B., Barry, R. A., Lieberburg, I., Prusiner, S. B., and Lingappa, V. R., Biogenesis and Transmembrane Orientation of the Cellular Isoform of the Scrapie Prion Protein, 7 MOL. CELL. BIOL. 914-920 (1987a); Hay, B., Prusiner, S. B., and Lingappa, V. R., Evidence for a Secretory Form of the Cellular Prion Protein, 26 BIOCHEM. 8110-8115 (1987b), all incorporated herein by reference). There is evidence that the relative proportions of the three topological variants is determined by as yet unidentified accessory proteins present during the translation process, as well as by a region of nine hydrophilic acids (the “stop transfer effector”) adjacent to the transmembrane domain of PrP (Hegde, R. S., Voigt, S., and Lingappa, V. R., Regulation of Protein Topology by Transacting Factors at the Endoplasmic Reticulum, 2 MOL. CELL. 85-91 (1998b); Lopez, C. D., Yost, C. S., Prusiner, S. B., Myers, R. M., and Lingappa, V. R., Unusual Topogenic Sequence Directs Prion Protein Biogenesis, 248 SCIENCE 226-229 (1990); Yost, C. S., Lopez, C. D., Prusiner, S. B., Myers, R. M., and Lingappa, V. R., Non-hydrophobic Extracytoplasmic Determinant of Stop Transfer in the Prion Protein, 343 NATURE 669-672 (1990), all incorporated herein by reference).

### SUMMARY OF THE INVENTION

The present invention is directed to the identification and further characterization of the structure of ^{Ctm}PrP and of mutant forms of PrP that are synthesized exclusively or in greater than wild type amounts with the ^{Ctm}PrP topology by mammalian cells as well as the use of compositions and methods to analyze and detect ^{Ctm}PrP with respect to the study and diagnosis of prion-caused diseases.

One embodiment of the present invention is a method for selective recognition of ^{Ctm}PrP in a mammal comprising identifying the presence of an uncleaved signal peptide on said ^{Ctm}PrP, including embodiments wherein said signal peptide has the sequence identified herein as the first 22 amino acids of SEQ ID: 5. The inventive method may also include utilizing antibodies that bind specifically to an uncleaved signal peptide region on ^{Ctm}PrP, an immunogenic fragment thereof, or the region of the cleavage site of the uncleaved signal peptide, to identify the ^{Ctm}PrP. Embodiments of the present invention include antibodies used in the inventive methods, including those that bind specifically to the uncleaved signal peptide, the first 22 amino acids of SEQ ID: 5, and the region of the cleavage site of the uncleaved signal peptide. Additional embodiments also include methods of pre and post-mortem diagnosis of prion-caused diseases in mammalian patients, including humans and livestock, by identifying the presence of an uncleaved signal peptide on ^{Ctm}PrP.

Another embodiment of the inventive method includes targeting a cysteine amino acid within the signal peptide, including such an acid at position 22, with sulfhydryl-reagents to identify the ^{Ctm}PrP.

Another embodiment of the present invention includes novel mammalian nucleic acid mutants comprising sequences that encode for a mutant form of PrP with an expressed topology of ^{Ctm}PrP greater than that of the wild type nucleic acid. Preferred embodiments of this aspect of the invention include isolated nucleic acids, cultured cells, expression vectors, methods of expression of mutant ^{Ctm}PrP, transgenic non-human animals, transgenic mice, and isolated ^{Ctm}PrP produced by such embodiments.

### BRIEF DESCRIPTION OF THE DRAWINGS

FIG. 1 shows an autoradiograph of an SDS-PAGE gel of in vitro translation products illustrating that mutations in the transmembrane region increase the proportion of ^{Ctm}PrP, and reveal that this form is slightly larger than ^{Sec}PrP.

FIG. 2 shows by immunoprecipitation of FLAG-tagged PrP that ^{Ctm}PrP contains an uncleaved signal peptide. Panel A shows a schematic of the immunoreactivity of FLAG-tagged ^{Ctm}PrP and ^{Sec}PrP. Panels B and C show autoradiograms of SDS-PAGE gels of in vitro translation products.

FIG. 3 shows that mutations in the signal sequence increase the proportion of ^{Ctm}PrP. Panels A and B show autoradiographs of SDS-PAGE gels of in vitro translation products, and Panel C shows a Western blot of cell lysates.

FIG. 4 shows an autoradiograph of an SDS-PAGE gel of lysates from metabolically labeled cells demonstrating that ^{Ctm}PrP contains a GPI anchor.

FIG. 5 shows a Western blot of cell lysates establishing that the oligosaccharide chains of ^{Ctm}PrP are sensitive to digestion with endoglycosidase H.

FIG. 6 shows immuofluorescence micrographs of cells expressing PrP which establish that ^{Ctm}PrP is retained in the ER.

FIG. 7 is a schematic drawing of three membrane topologies of PrP.

FIG. 8 is SEQ ID: 1, a mouse nucleic acid sequence encoding for a wild-type PrP polypeptide.

FIG. 9 is SEQ ID: 2, a mouse nucleic acid sequence encoding for an L9R mutant PrP polypeptide.

FIG. 10 is SEQ ID: 3, a mouse nucleic acid sequence encoding for a 3AV mutant PrP polypeptide.

FIG. 11 is SEQ ID: 4, a mouse nucleic acid sequence encoding for the L9R/3AV double mutant PrP polypeptide.

FIG. 12 is SEQ ID: 5, an amino acid sequence of the wild-type mouse PrP polypeptide.

FIG. 13 is SEQ ID: 6, an amino acid sequence of the L9R mutant mouse PrP polypeptide.

FIG. 14 is SEQ ID: 7, an amino acid sequence of the 3AV mutant mouse PrP polypeptide.

FIG. 15 is SEQ ID: 8, an amino acid sequence of the L9R-3AV double mutant mouse PrP polypeptide.

### DETAILED DESCRIPTION OF THE PREFERRED EMBODIMENTS

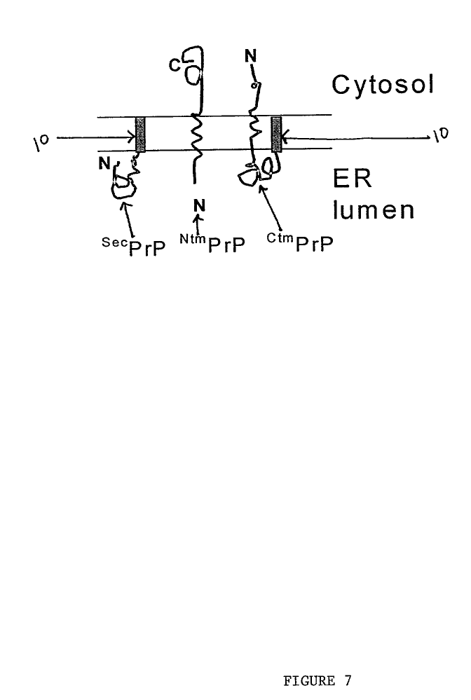

The topology of the ^{Sec}Prp, ^{Ntm}Prp and ^{Ctm}PrP variants is shown schematically in FIG. 7. In the secreted form of PrP (“^{Sec}Prp”), which is the predominant form under normal circumstances, the polypeptide chain lies entirely in the endoplasmic reticulum (“ER”) lumen equivalent to the exofacial surface. ^{Ntm}Prp and ^{Ctm}PrP result from membrane insertion of the central hydrophobic sequence, so that the only N-terminus or C-terminus, respectively, of the polypeptide chain lie in the ER lumen. FIG. 7 also illustrates the presence of a glycolipid anchor structure 10 on ^{Sec}Prp and ^{Ctm}PrP, further discussed below.

Several pieces of evidence have implicated ^{Ctm}PrP in the pathogenesis of prion diseases. First, transgenic mice were created that express PrP molecules carrying mutations in or near the transmembrane domain that favor formation of ^{Ctm}PrP (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE. 827-834 (1998a); Hegde, R. S., Tremblay, P., Groth, D., DeArmond, S. J., Prusiner, S. B., and Lingappa, V. R., Transmissible and Genetic Prion Diseases Share a Common Pathway of Neurodegeneration, 402 NATURE 822-826 (1999), both incorporated herein by reference). Mice that produced ^{Ctm}PrP above a threshold level developed a spontaneous neurological disease with scrapie-like features, but without detectable PrP^{Sc}. In addition, when these mice were inoculated with scrapie prions, the amount of PrP^{Sc} that accumulated was inversely related to the amount of ^{Ctm}PrP present, indicating that ^{Ctm}PrP rather than PrP^{Sc} may be the proximate cause of neurodegeneration (Hegde, R. S., Tremblay, P., Groth, D., DeArmond, S. J., Prusiner, S. B., and Lingappa, V. R. Transmissible and genetic prion diseases share a common pathway of neurodegeneration. 402 NATURE. 822-826 (1999)). Finally, after scrapie inoculation of mice that carried a wild-type hamster PrP transgene that served as a reporter of ^{Ctm}PrP formation, ^{Ctm}PrP was found to accumulate during the course of the infection (Hegde, R. S., Tremblay, P., Groth, D., DeArmond, S. J., Prusiner, S. B., and Lingappa, V. R. Transmissible and genetic prion diseases share a common pathway of neurodegeneration. 402 NATURE. 822-826 (1999)). Taken together, these data have been interpreted to suggest that ^{Ctm}PrP is the direct cause of neurodegeneration in familial and infectious prion diseases, and that PrP^{Sc} acts indirectly by increasing the amount of ^{Ctm}PrP.

As shown in FIG. 7, ^{Ctm}PrP has the topology of a type II transmembrane protein (C-terminus on the exofacial side of the bilayer), and it obeys the “positive inside” rule in which there is a preponderance of positively charged residues on the cytoplasmic side of the membrane-spanning sequence (von Heijne, G., Control of Topology and Mode of Assembly of a Polytopic Membrane Protein by Positively Charged Residues, 341 NATURE 456-458 (1989), incorporated herein by reference). However, ^{Ctm}PrP is unusual in that it contains an uncleaved, N-terminal signal peptide identified in accordance with the present invention. Most type II proteins contain an internal signal-anchor sequence that serves both to initiate translocation and to anchor the polypeptide chain in the lipid bilayer (Denzer, A. J., Nabholz, C. E., and Spiess, M., Transmembrane Orientation of signal-anchor Proteins is Affected by the Folding State But Not the Size of the N-terminal Domain, 14 EMBO. J. 6311-6317 (1995); Gafvelin, G., Sakaguchi, M., Andersson, H., and von Heijne, G., Topological Rules for Membrane Protein Assembly in Eukaryotic Cells, 272 J. BIOL. CHEM. 6119-6127 (1997), both incorporated herein by reference). A few type II proteins have an uncleaved, N-terminal signal sequence, but unlike the case of ^{Ctm}PrP, this sequence serves as a membrane anchor (Ozols, J., Determination of Lumenal Orientation of Microsomal 50-kDa Esterase/N-deacetylase, 37 BIOCHEMISTRY 10336-10344 (1998), incorporated herein by reference). The retention of the N-terminal signal peptide on ^{Ctm}PrP can be rationalized by the fact that the N-terminus of the polypeptide chain does not enter the ER lumen where signal peptidase is located. In contrast, the signal sequence is cleaved from ^{Sec}PrP and ^{Ntm}Prp, whose N-termini lie on the lumenal side of the membrane.

^{Ctm}PrP is unusual in one other respect, which is the presence of a C-terminal GPI anchor in addition to the transmembrane anchor. ^{Ctm}PrP contains a GPI anchor shown by labeling cells with [³H]palmitate, as well as by PIPLC treatment of PrP translated in vitro followed by Triton X-114 phase partitioning (Stewart, R. S., and Harris, D. A. Most Pathogenic Mutations do not Alter the Membrane Topology of the Prion Protein. 276 J. BIOL. CHEM. 2212-2220 (2001). This dual mode of membrane attachment has been described in only a few other proteins (Hitt, A. L., Lu, T. H., and Luna, E. J., Ponticulin is an Atypical Membrane Protein, 126 J. CELL BIOL. 1421-1431 (1994); Howell, S., Lanctot, C., Boileau, G., and Crine, P., A Cleavable N-terminal Signal Peptide is Not a Prerequisite for the Biosynthesis of Glycosylphosphatidylinositol-anchored Proteins, 269 J. BIOL. CHEM. 16993-16996 (1994); Koster, B., and Strand, M., Schistosoma mansoni: Sm23is a Transmembrane Protein That Also Contains a Glycosylphosphatidylinositol Anchor, 310 ARCH. BIOCHEM. BIOPHYS. 108-117 (1994), all incorporated herein by reference). The presence of a GPI anchor on ^{Ctm}PrP is consistent with the fact that anchor addition occurs on the lumenal side of the ER membrane after cleavage of a C-terminal segment of the polypeptide chain (Udenfriend, S., and Kodukula, K., How Glycosylphosphatidylinositol-Anchored Membrane Proteins are Made, 64 ANNU. REV. BIOCHEM. 563-591 (1995), incorporated herein by reference).

The data suggests a model in which the membrane orientation of PrP is determined by competition during the translation process between two conflicting topological determinants in the polypeptide chain: an N-terminal signal sequence (residues 1-22) that directs translocation of the N-terminus of the polypeptide chain across the membrane to produce ^{Sec}PrP or ^{Ntm}PrP; and a central hydrophobic domain (residues 111-134) that acts as a type II signal-anchor sequence, directing translocation of the C-terminus across the membrane to produce ^{Ctm}PrP. (Hegde, R. S., and Lingappa, V. R., Regulation of Protein Biogenesis at the Endoplasmic Reticulum Membrane, 9 TRENDs CELL BIOL. 132-137 (1999), incorporated herein by reference.) In this model, the effects of mutations on the membrane orientation of PrP (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE. 827-834 (1998a); Hegde, R. S., Tremblay, P., Groth, D., DeArmond, S. J., Prusiner, S. B., and Lingappa, V. R. Transmissible and genetic prion diseases share a common pathway of neurodegeneration. 402 NATURE. 822-826 (1999)) can be understood in terms of how they affect the relative functional strength of these two topological domains. Mutations within or near the central, hydrophobic domain either enhance (3AV, N107I, K109I/H110I, A116V) or diminish (G122P) the efficacy of the internal signal-anchor sequence, thereby either increasing or decreasing the proportion of ^{Ctm}PrP. The “stop transfer effector” (residues 103-111), which modulates formation of ^{Ctm}PrP, is also thought to act by altering the action of the adjacent signal-anchor region (Lopez, C. D., Yost, C. S., Prusiner, S. B., Myers, R. M., and Lingappa, V. R. Unusual topogenic sequence directs prion protein biogenesis. 248 SCIENCE. 226-229 (1990); Yost, C. S., Lopez, C. D., Prusiner, S. B., Myers, R. M., and Lingappa, V. R. Non-hydrophobic extracytoplasmic determinant of stop transfer in the prion protein. 343 NATURE. 669-672 (1990)). In contrast, the novel L9R mutation discussed herein weakens the translocation activity of the N-terminal signal peptide by introducing a charged residue into the hydrophobic core of the sequence, thus increasing the proportion of ^{Ctm}PrP.

The novel inventive combination of mutations in both the signal and signal-anchor domains (L9R/3AV), yields a synergistic effect that completely shifts the topology of PrP to the ^{Ctm}PrP form. Competitive interactions between an N-terminal signal sequence and an internal signal-anchor sequence have also been observed in model chimeric proteins (Goder, V., Bieri, C., and Spiess, M., Glycosylation Can Influence Topogenesis of Membrane Proteins and Reveals Dynamic Reorientation of Nascent Polypeptides Within the Translocon, 147 J. CELL BIOL. 257-266 (1999), incorporated herein by reference), indicating that the adoption of alternate membrane topologies by a single polypeptide chain is not a unique feature of PrP. The precise mechanisms by which signal and signal-anchor sequences interact during the translation process remain to be investigated, although there is evidence that the two determinants compete within the translocon itself rather than at the level of binding to signal recognition particle (Goder, V., Bieri, C., and Spiess, M. Glycosylation Can Influence Topogenesis of Membrane Proteins and Reveals Dynamic Reorientation of Nascent Polypeptides Within the Translocon, 147 J. CELL BIOL. 257-266 (1999); Hegde, R. S., Voigt, S., and Lingappa, V. R. Regulation of Protein Topology by Trans-acting Factors at the Endoplasmic Reticulum, 2 MOL. CELL. 85-91 (1998b)). Consistent with this suggestion, PrP in which the N-terminal signal sequence has been deleted is not translocated into microsomes at all, indicating that the signal-anchor sequence is not by itself competent for binding to the signal recognition particle and targeting to the ER.

Previously identified mutations in the transmembrane domain increase the proportion of ^{Ctm}PrP to at most 30-40% of the total PrP chains after in vitro translation (Hegde, R. S., Mastrianni, J. A., Scott, M. R., Defea, K. A., Tremblay, P., Torchia, M., DeArmond, S. J., Prusiner, S. B., and Lingappa, V. R. A Transmembrane Form of the Prion Protein in Neurodegenerative Disease, 279 SCIENCE 827-834 (1998a); Hegde, R. S., Tremblay, P., Groth, D., DeArmond, S. J., Prusiner, S. B., and Lingappa, V. R. Transmissible and Genetic Prion Diseases Share a Common Pathway of Neurodegeneration, 402 NATURE 822-826 (1999)), and to only 2% after transfection of cultured cells (Stewart, R. S., and Harris, D. A., Most Pathogenic Mutations Do Not Alter the Membrane Topology of the Prion Protein, 276 J. BIOL. CHEM. 2212-2220 (2001)), with the remainder of the molecules being primarily ^{Sec}PrP. In contrast, the novel double mutation L9R/3AV results in virtually 100% of the molecules assuming a ^{Ctm}PrP orientation. Thus, this embodiment of the invention allows the study of the biological features of ^{Ctm}PrP in the absence of the two other topological variants of PrP. Using the inventive L9R/3AV PrP, it can be seen that ^{Ctm}PrP fails to reach the cell surface after synthesis, and is retained primarily in the ER. This localization may result from recognition of ^{Ctm}PrP as a misfolded substrate by the ER quality control machinery (Ellgaard, L., Molinari, M., and Helenius, A. Setting the standards: quality control in the secretory pathway. 286 SCIENCE. 1882-1888 (1999)).

A previous report concluded that ^{Ctm}PrP transits beyond the ER, based on the endoglycosidase H-resistance of PrP from the brains of transgenic mice that express the 3AV or K109I/H110I mutations (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE. 827-834 (1998a)). However, only 20-30% of the PrP in these animals is actually ^{Ctm}PrP, and biochemical characterization of these molecules in the presence of excess ^{Sec}PrP would have been problematic. Interestingly, Zanusso et al. found that PrP carrying a stop codon at position 145, a mutation described in a Japanese patient with a Gerstmann-Straussler-like syndrome, retained the N-terminal signal peptide and was rapidly degraded by the proteasome (Zanusso, G., Petersen, R. B., Jin, T., Jing, Y., Kanoush, R., Ferrari, S., Gambetti, P., and Singh, N., Proteasomal Degradation and N-terminal Protease Resistance of the Codon 145 Mutant Prion Protein, 274 J. BIOL. CHEM. 23396-23404 (1999), incorporated herein by reference). Unlike L9R/3AV PrP, however, this mutant was partially secreted. These results suggest that alterations of the C-terminal part of PrP beyond the signal-anchor sequence can produce a topological variant with the characteristics of both ^{Ctm}PrP and ^{Sec}PrP.

Mutant L9R/3AV PrP is detergent-insoluble, possibly because of the presence of the hydrophobic, N-terminal signal peptide on the cytoplasmic domain, but it is not resistant to digestion with even low concentrations of PK in detergent solution, a cardinal feature of PrP^{Sc} from infectious, familial and sporadic cases of prion disease. Similarly, Hegde et al. have not observed a PrP 27-30 fragment after digestion of PrP molecules carrying other ^{Ctm}PrP-favoring mutations (although small amounts of slightly smaller fragment are produced under mild digestion conditions) (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE. 827-834 (1998a)). Whether ^{Ctm}PrP and PrP^{Sc} contribute independently to neurodegeneration, or whether they form part of a common biochemical pathway remains to be determined. Expression of L9R/3AV PrP in the inventive transgenic mice, which would be predicted to produce a severe neurological illness without PrP^{Sc}, may help to further illuminate the role of ^{Ctm}PrP in prion diseases.

Although specific examples and embodiments of the present invention are identified herein and below, they are not intended to be and shall not be interpreted to be limiting of the invention.

A. Definitions and Methods

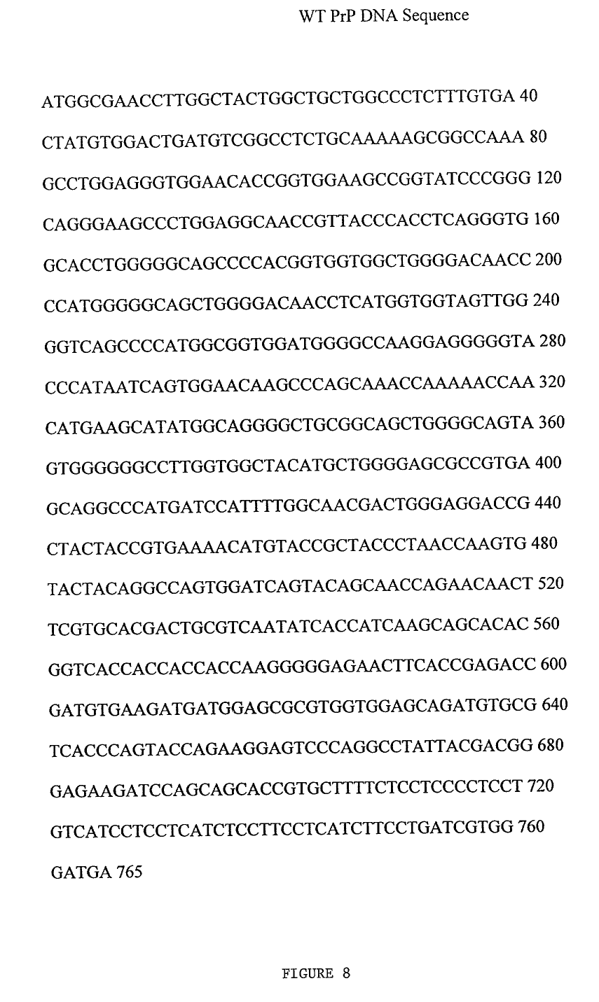

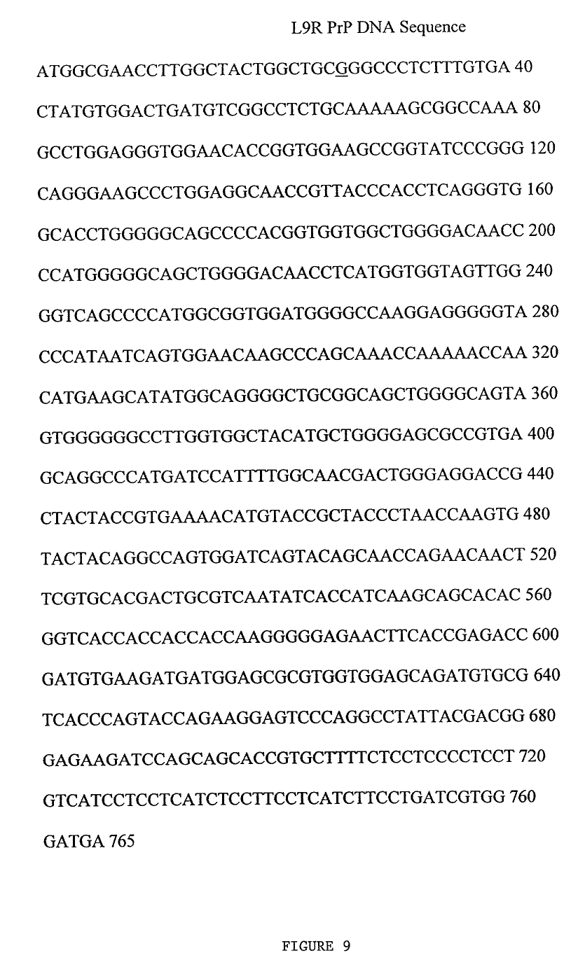

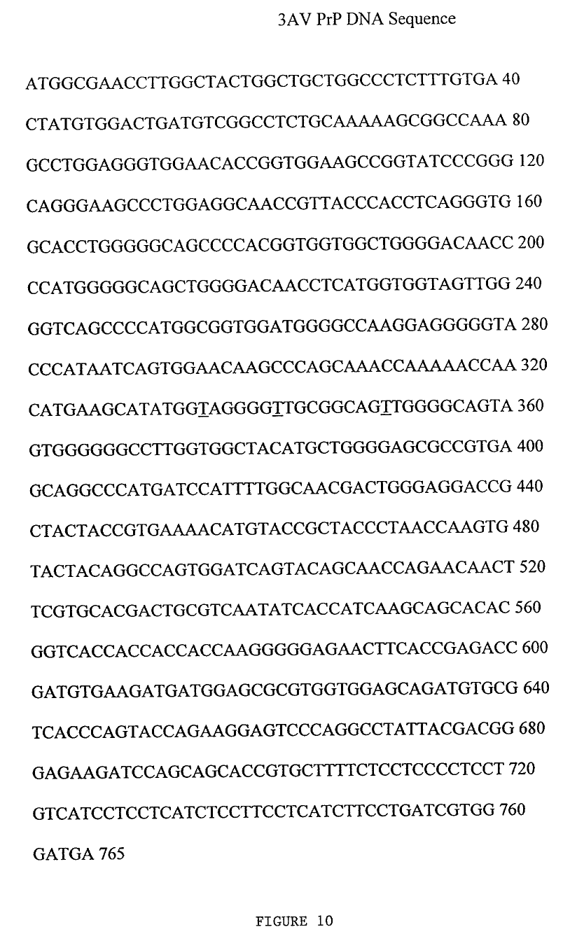

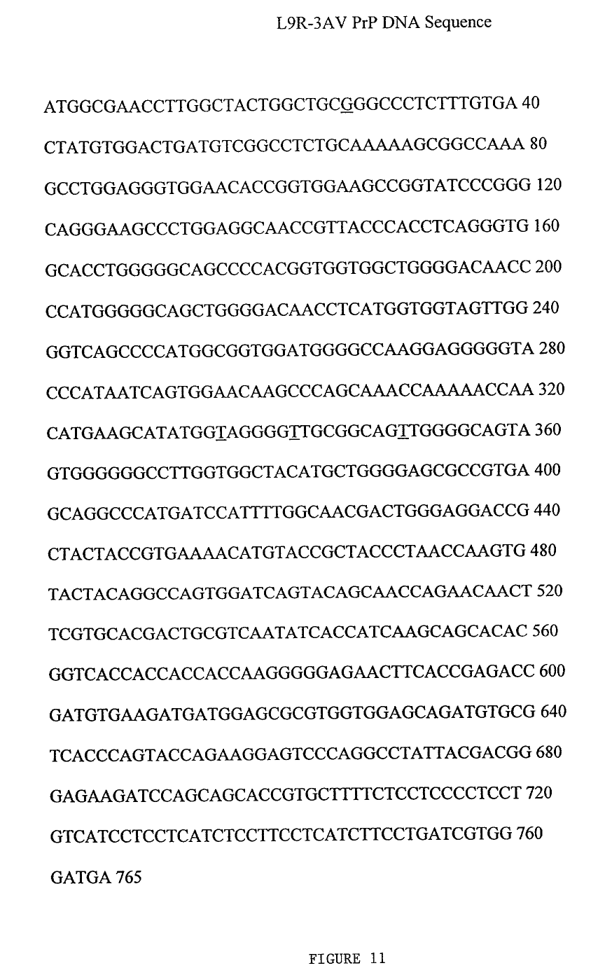

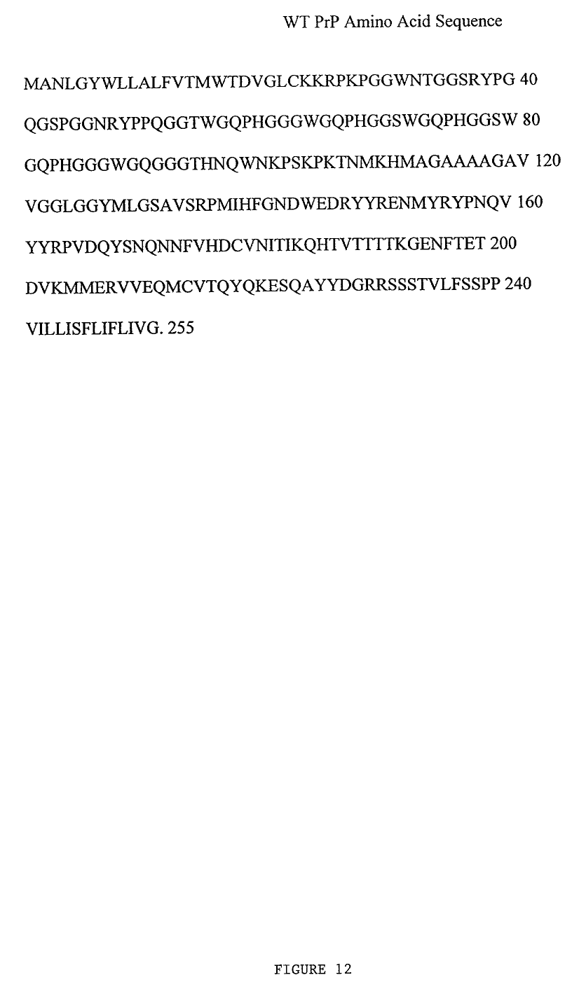

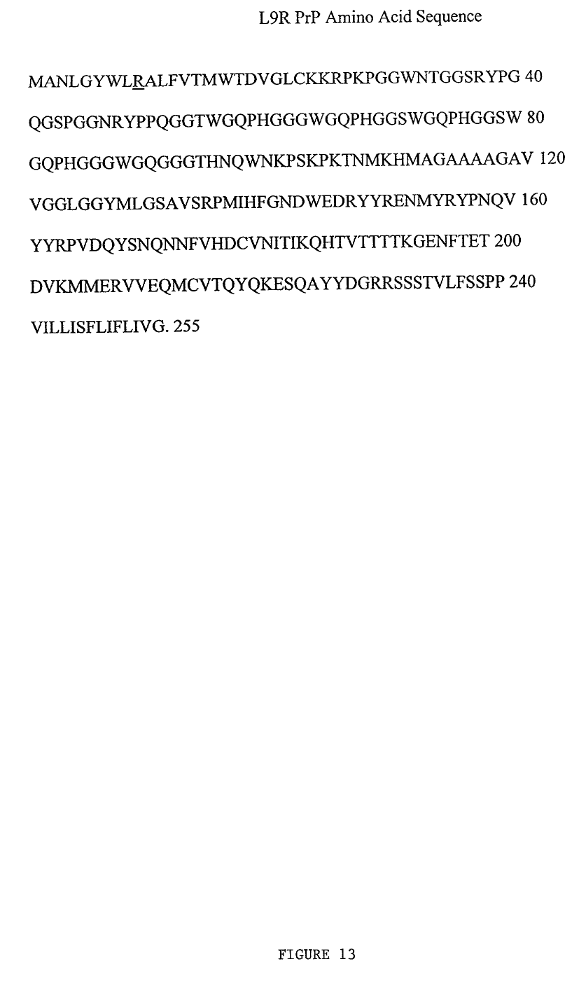

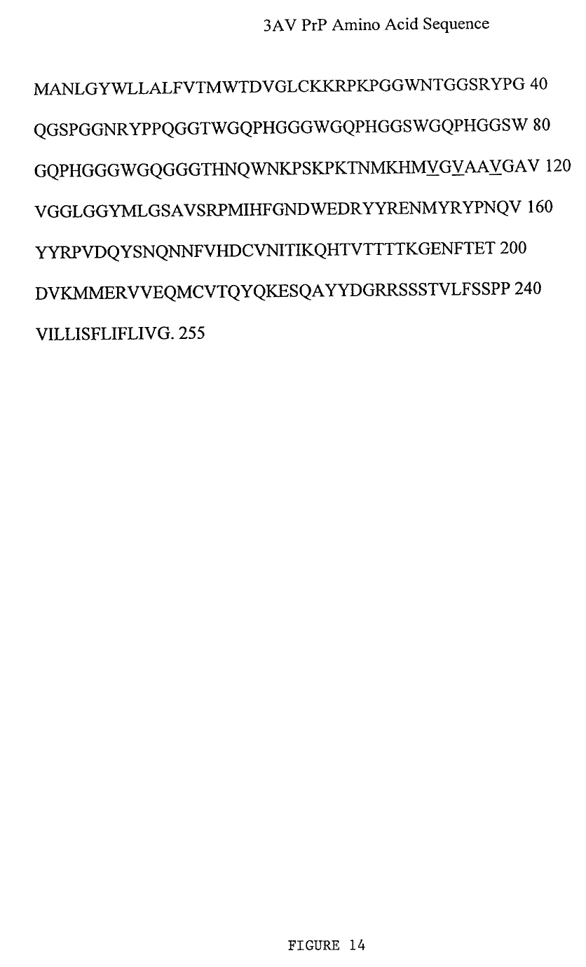

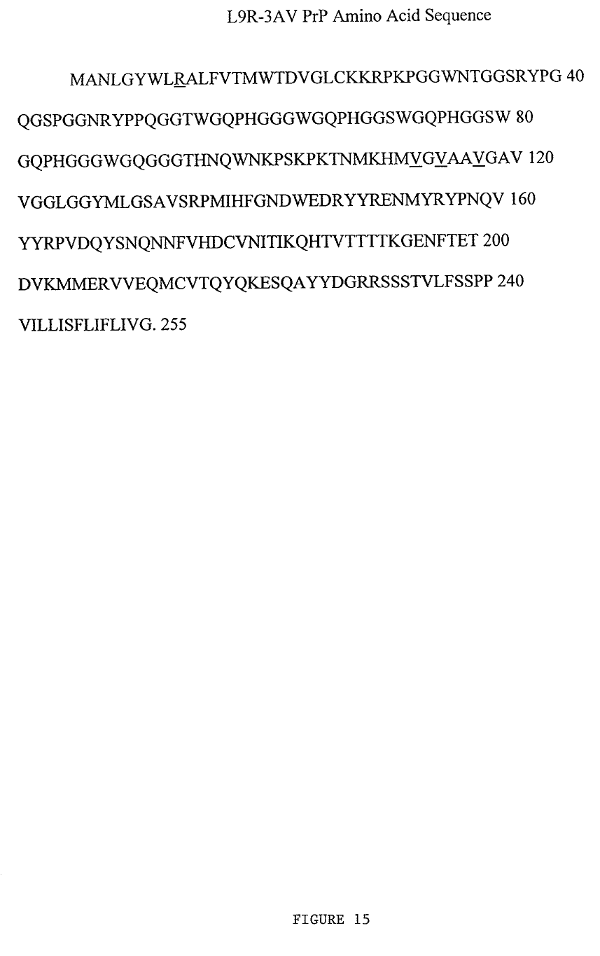

PrP plasmids and mRNA synthesis. All mouse PrP cDNAs were cloned into the vector pcDNA3 (Invitrogen), and carried an epitope tag for monoclonal antibody 3F4 created by changing residues 108 and 111 to methionine. As described below, the following four mutations were introduced into the wild-type PrP cDNA (FIG. 8, SEQ ID: 1) using PCR as described previously (Lehmann, S., and Harris, D. A. A mutantprion protein displays an aberrant membrane association when expressed in cultured cells. 270 J. BIOL. CHEM. 24589-24597 (1995)): L9R, A116V, 3AV (A→V at 112, 114 and 117), and L9R/3AV. The FLAG epitope (DYKDDDDK) was inserted between residues 22 and 23 using PCR. Plasmids were linearized with Xba I and gel-purified. In vitro transcriptions were performed with the mMessage mMachine T7 kit (Ambion). Nucleic acid sequences for the mouse L9R, 3AV and double mutant L9R/3AV DNA are given in FIGS. 9, 10 and 11 as SEQ IDs: 2, 3, and 4 respectively. Amino acid sequences for the mouse WT, L9R, 3AV and L9R-3AV mutants are shown in FIGS. 12, 13, 14 and 15 as SEQ IDs: 5, 6, 7, and 8, respectively.

In vitro translation. Messenger RNAs were translated in the presence of [³⁵S]methionine (Promix, Amersham) using rabbit reticulocyte lysate (Promega) as directed by the manufacturer, except that the final lysate concentration was 50%. Translation reactions also contained microsomal membranes prepared from canine pancreas (Promega) or from murine BW5174.3 thymoma cells (Vidugiriene, J., and Menon, A. K., Soluble Constituents of the ER Lumen are Required for GPI Anchoring of a Model Protein, 14 EMBO. J. 4686-4694 (1995), incorporated herein by reference). To detect protease-protected products, 5 microliter aliquots of translation reactions were incubated in a final volume of 50 μl with 100 μg/ml of PK (Boehringer Mannheim) in 50 mM Tris-HCl (pH 7.5) and 1 mM CaCl₂ for 60 min at 4° C., followed by addition of 5 mM PMSF to terminate digestion. Some digestion reactions also contained 0.5% Triton-X 100 to solubilize membranes. Samples were then analyzed by SDS-PAGE and autoradiography.

Immunoprecipitation. Aliquots of the translation reactions were boiled in the presence of 1% SDS for 5 min to denature the proteins, and were then diluted with 10 volumes of RIPA buffer (50 mM Tris-HCl [pH 7.5], 150 mM NaCl, 0.1% Triton X-100, 0.5% sodium deoxycholate, 0.1% SDS) containing 1 mM CalCl₂ plus protease inhibitors (1 μg/ml pepstatin A, 1 μg/ml leupeptin, 5 mM PMSF). Anti-PrP antibody P45-66 (Lehmann, S., and Harris, D. A. A mutant prion protein displays an aberrant membrane association when expressed in cultured cells. 270 J. BIOL. CHEM. 24589-24597 (1995)), or anti-FLAG monoclonal antibodies M1 or M2 (Sigma), were added and samples incubated on ice for 90 min. Protein A-Sepharose beads (for P45-66 and M1) or protein G-agarose beads (for M2) were added, and samples were rotated at 4° C. for 30 min. Beads were collected by low-speed centrifugation and washed three times with RIPA buffer plus 1 mM CaCl₂, after which proteins were eluted with sample buffer, and analyzed by SDS-PAGE.

Transfected cells. BHK and CHO cells, including the inventive L9R/3AV double-mutant cells, were maintained in minimal essential medium (“MEM”) supplemented with 10% fetal calf serum, non-essential amino acids, and penicillin/streptomycin. Transfections were performed with Lipofectamine (BRL) according to the manufacturer's instructions. Cells were harvested 24 hrs after transfection by scraping or by brief trypsinization, rinsed twice with PBS, and resuspended in 0.25 M sucrose, 10 mM HEPES (pH 7.4), 1 g/ml pepstatin A, 1 g/ml leupeptin. Cells were disrupted by 10 passages through silastic tubing (0.3 mm i.d.) connecting two syringes with 27-gauge needles. A post-nuclear supernatant was prepared by centrifugation at 2,500×g for 2 min. PK protection assays were performed by incubating post-nuclear supernatants in 50 mM Tris-HCl (pH 7.5), 250 g/ml PK, and in some cases 0.5% Triton X-100. After 60 min at 22° C., digestion was terminated by addition of 5 mM PMSF and samples were treated with PNGase F (New England Biolabs) and analyzed by Western blotting with anti-PrP antibody 3F4 (Bolton, D. C., Seligman, S. J., Bablanian, G., Windsor, D., Scala, L. J., Kim, K. S., Chen, C. M., Kascsak, R. J., and Bendheim, P. E., Molecular Location of a Species-Specific Epitope on the Hamster Scrapie Agent Protein, 65 J. VIROL. 3667-3675 (1991), incorporated herein by reference).

To test the glycosidase sensitivity of PrP, lysates of transfected cells prepared in 0.5% Triton X-100, 0.5% deoxycholate, 50 mM Tris-HCl (pH 7.5) were treated with endoglycosidase H (New England Biolabs) or PNGase F according to the manufacturer's directions prior to methanol precipitation and Western blotting with 3F4.

GPI anchor analysis. Transiently transfected cells were labeled for 16 hr with either 3.5 mCi/ml [³H]palmitic acid (American Radiochemical Corp.) or 250 μCi/ml [³⁵S]methionine. After lysis in 0.5% SDS, 50 mM Tris-HCl (pH 7.5), samples were diluted with 5 volumes of 0.5% Triton X-100, 50 mM Tris-HCl (pH 7.5) and incubated for 16 hr at 37° C. with PNGase F in the presence or absence of PIPLC (1 unit/ml) from B. thuringiensis. PrP was then immunoprecipitated with 3F4 antibody and analyzed by SDS-PAGE.

Immunofluorescence microscopy. Transiently transfected cells grown on glass coverslips were fixed for 1 hr in 4% paraformaldehyde in PBS, and then permeabilized for 2 min in 0.5% Triton-X-100 in PBS. After treatment for 30 min in 2% goat serum/PBS (blocking solution), cells were incubated for 1 hr with primary antibodies in blocking solution (P45-66 and mouse anti-protein disulfide isomerase [Stressgen]), washed, and then incubated for 1 hr with fluorescently labeled secondary antibodies in blocking solution (Alexa-488-coupled anti-rabbit IgG and Alexa-594-coupled anti-mouse IgG from Molecular Probes). Coverslips were then mounted in 50% glycerol/PBS, and viewed with a Zeiss Axioplan fluorescence microscope equipped with a Bio-Rad MRC1024 laser confocal scanning system. To selectively visualize surface PrP, living cells were stained with 3F4 antibody in Opti-MEM (BRL) plus 2% goat serum, washed, fixed in 4% paraformaldehyde, and then incubated with Alexa-488-coupled anti-mouse IgG.

B. Identification of an Uncleaved Signal Peptide on ^{Ctm}PrP.

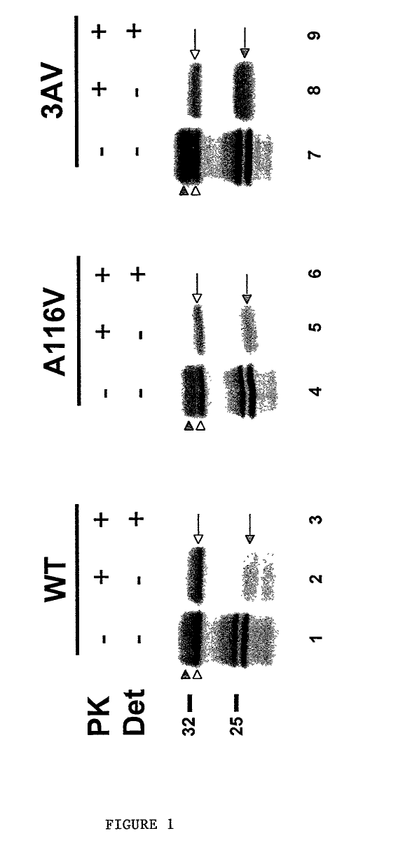

One embodiment of the present invention provides for the identification of a novel uncleaved signal peptide on ^{Ctm}PrP. FIG. 1, lanes 1, 4 and 7, shows that when PrP mRNA is translated in vitro using rabbit reticulocyte lysate supplemented with canine pancreatic microsomes, products of ˜32 kDa and ˜25 kDa are synthesized, corresponding to core-glycosylated and untranslocated/unglycosylated PrP, respectively. Messenger RNA encoding wild-type (WT), A116V or 3AV PrP was translated in rabbit reticulocyte lysate supplemented with canine pancreatic microsomes. Aliquots of the reaction were then incubated with (lanes 2, 3, 5, 6, 8, 9) or without (lanes 1, 4, 7) PK in the presence (lanes 3, 6, 9) or absence (lanes 1, 2, 4, 5, 7, 8) of Triton X-100 (Det). Samples were then analyzed by SDS-PAGE and autoradiography. Incubating microsomes with PK cleaved off the cytoplasmically exposed domains of newly synthesized PrP molecules resulting in the appearance of two protease-protected species (FIG. 1, lanes 2, 5, 8): a 32 kDa form (^{Sec}PrP) that corresponds to intact, fully translocated chains (shown as white arrows in FIG. 1), and a 24 kDa fragment that corresponds to the transmembrane and lumenal domains of ^{Ctm}PrP (shown as shaded arrows in FIG. 1). The latter fragment is distinct from untranslocated/unglycosylated PrP which has a slightly larger molecular size, and is not present in lanes 2, 5, and 8 because it is completely degraded by the protease. As reported previously (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE. 827-834 (1998a); Hegde, R. S., Tremblay, P., Groth, D., DeArmond, S. J., Prusiner, S. B., and Lingappa, V. R. Transmissible and genetic prion diseases share a common pathway of neurodegeneration. 402 NATURE. 822-826 (1999)), the presence of either of two mutations (A116V or the 3AV mutation) in the transmembrane domain significantly increased the proportion of ^{Ctm}PrP (FIG. 1, lanes 5 and 8). No PrP was detected after PK treatment in the presence of Triton X-100 detergent which disrupts the microsomal membrane (FIG. 1, lanes 3, 6, 9), confirming that the 32 and 24 kDa fragments do not represent intrinsically protease-resistant portions of the molecule.

A 33 kDa glycosylated product that was present before PK digestion could be resolved from the 32 kDa band corresponding to ^{Sec}Prp (FIG. 1, lanes 1, 4, 7). The amount of this 33 kDa species (indicated by shaded arrowheads) correlated with the amount of the 24 kDa ^{Ctm}PrP fragment (shown as shaded arrows) produced after PK digestion: it was present in largest amounts for 3AV, at intermediate levels for A116V, and in smallest amounts for wild-type PrP. This observation suggested that the 33 kDa species corresponded to full-length ^{Ctm}PrP, which then gave rise to a 24 kDa protected fragment after PK digestion.

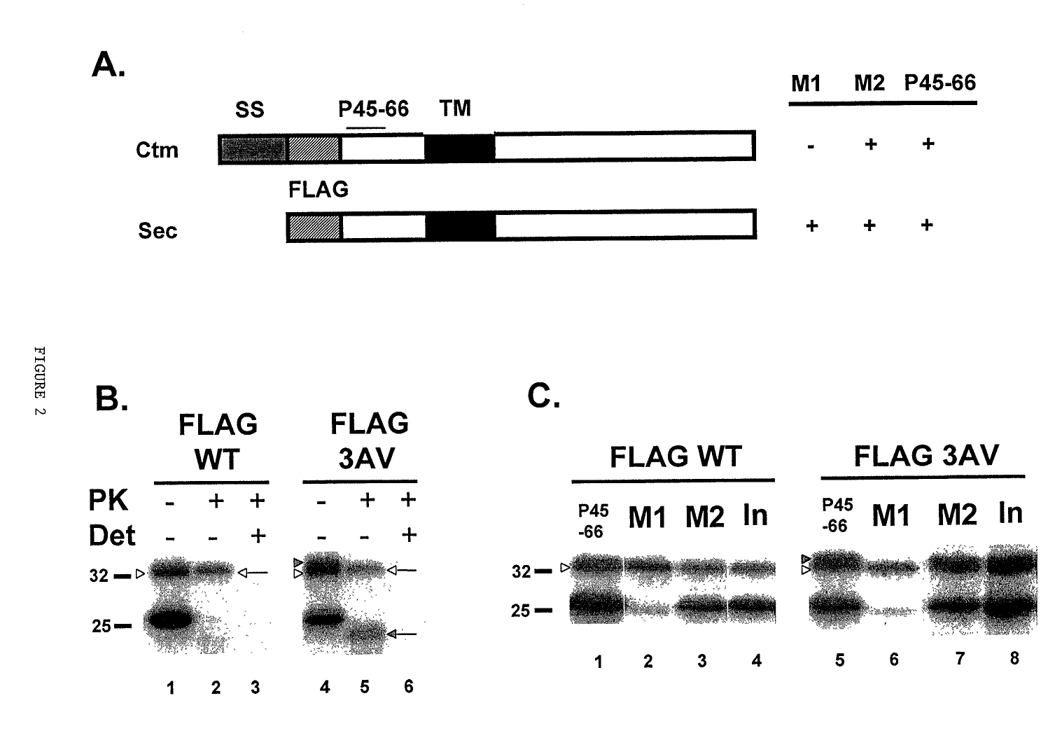

FIG. 2A is a schematic of the immunoreactivity of FLAG-tagged ^{Ctm}PrP and ^{Sec}Prp showing the signal sequence (“SS”) and the transmembrane domain (“TM”). Wild-type and 3AV versions of PrP were constructed that contained an eight amino acid FLAG epitope (DYKDDDDK) inserted at the signal peptide cleavage site, between residues 22 and 23 (FIG. 2A). FIG. 2B shows the in vitro translation and PK protection assays of FLAG-tagged wild-type (“WT”) or 3AV PrP which was performed as those shown in FIG. 1, except that microsomes were from murine BW5174.3 thymoma cells. The white and shaded arrowheads indicate, respectively, the positions of ^{Sec}Prp and ^{Ctm}PrP prior to protease digestion (lanes 1 and 4); it is not possible to completely separate the FLAG-tagged versions of these two species. The white and shaded arrows indicate, respectively, the protease-protected forms of ^{Sec}Prp and ^{Ctm}PrP (lanes 2 and 5). Only Se^{Ctm}PrP is visible for WT PrP. FIG. 2C illustrates that FLAG-WT and FLAG-3AV PrPs were synthesized by in vitro translation, and were immunoprecipitated with anti-PrP antibody P45-66 (lanes 1 and 5), anti-FLAG antibody M1 (lanes 2 and 6), or anti-FLAG antibody M2 (lanes 3 and 7). Lanes 4 and 8 show samples prior to immunoprecipitation. Note that ^{Sec}PrP (white arrowheads) but not ^{Ctm}PrP (shaded arrowhead) is immunoprecipitated with M1, while both forms are immunoprecipitated with P45-66 and M2.

The M1 antibody used recognizes the FLAG epitope only if it displays a free N-terminus (Prickett, K. S., Amberg, D. C., and Hopp, T. P. A Calcium-Dependent Antibody for Identification and Purification of Recombinant Proteins, 7 BIOTECHNIQUES 580-589 (1989), incorporated herein by reference). Thus, FLAG-tagged PrP will only react with M1 if the signal peptide has been cleaved. Antibody M2 which recognizes the FLAG epitope regardless of its sequence context, as well as antibody P45-66 which reacts with the octapeptide repeat region of PrP (Lehmann, S., and Harris, D. A. A mutant prion protein displays an aberrant membrane association when expressed in cultured cells. 270 J. BIOL. CHEM. 24589-24597 (1995)) were used as controls. Microsomes derived from BW5174.3 murine thymoma cells were used because they are efficient at attaching GPI anchors to newly synthesized polypeptide chains, in contrast to microsomes from canine pancreas (Vidugiriene, J., and Menon, A. K. Soluble constituents of the ER lumen are required for GPI anchoring of a model protein. 14 EMBO J. 4686-4694 (1995)). In addition, introduction of the FLAG epitope has relatively little effect on the proportions of ^{Sec}PrP and ^{Ctm}PrP translated with thymoma microsomes, whereas it increases the amount of ^{Ctm}PrP produced with pancreatic microsomes (data not shown). It had been previously shown that introduction of a FLAG epitope at the signal peptide cleavage site of PrP did not interfere with signal peptide removal, oligosaccharide addition, or the ability of the protein to be converted to PrP^{Sc} in transgenic mice (Telling, G. C., Tremblay, P., Torchia, M., DeArmond, S. J., Cohen, F. E., and Prusiner, S. B., N-terminally Tagged Prion Protein Supports Prion Propagation in Transgenic Mice, 6 PROTEIN Sci. 825-833 (1997), incorporated herein by reference).

FLAG-tagged PrP, like its untagged counterpart, gives rise to protected products corresponding to ^{Sec}PrP (32 kDa) and ^{Ctm}PrP (24 kDa) after PK digestion of microsomes and the amount of the ^{Ctm}PrP product is increased by the presence of the 3AV mutation (FIG. 2B, lanes 2 and 5). Before PK digestion, there was a broad band at 32-33 kDa which was especially apparent for 3AV PrP (FIG. 2B, lane 4), and which represents a combination of the 32 and 33 kDa species that we had seen after translation with pancreatic microsomes (FIG. 1); these two products are not completely resolved from each other in this experiment, probably because of the small increment in size contributed by the FLAG epitope. When undigested samples were subjected to immunoprecipitation, the M1 antibody reacted with the lower part of the 32-33 kDa band corresponding to ^{Sec}Prp, but not the upper portion of the band corresponding to what is believed to be full-length ^{Ctm}rPrP (FIG. 2C, compare lanes 6 and 8). The entire band (representing both ^{Sec}PrP and full-length ^{Ctm}PrP) was immunoprecipitated by antibodies P45-66 and M2 (FIG. 2C, lanes 5 and 7). This result indicates that while the signal peptide has been removed from ^{Sec}PrP, it is still present in ^{Ctm}PrP. The 25 kDa unglycosylated form of PrP, which represents untranslocated molecules whose signal peptide would still be present, was not recognized by M1 (FIG. 2C, lanes 2 and 5) and 6), a result which serves as a control for the specificity of the M1 antibody.

Confirmation that ^{Ctm}PrP contains an uncleaved signal peptide was established by labeling in vitro translation products with either [³H]leucine or [³⁵S]methionine. Radiolabeling of the 33 kDa band corresponding to ^{Ctm}PrP was much greater with [³H]leucine than with [³⁵S]methionine, consistent with the fact that the N-terminal signal sequence contains 5 leucine residues while the rest of the polypeptide chain (excluding the C-terminal GPI signal sequence) contains only 2 leucine residues.

C. Creation of a Novel Double Mutant that is Synthesized Exclusively as ^{Ctm}PrP.

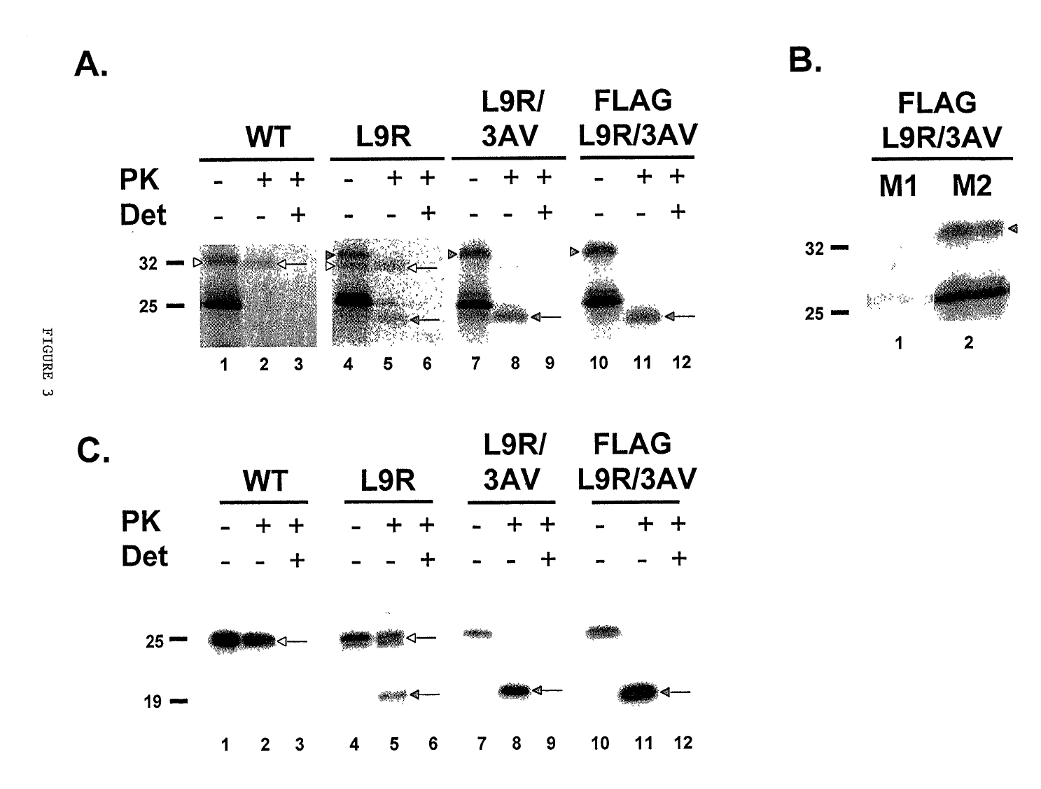

Previously identified mutations that have been found to alter the proportion of ^{Ctm}PrP are all are localized within or adjacent to the transmembrane domain (Hegde, R. S., Mastrianni, J. A., Scott, M. R., Defea, K. A., Tremblay, P., Torchia, M., DeArmond, S. J., Prusiner, S. B., and Lingappa, V. R. A transmembrane form of the prion protein in neurodegenerative disease. 279 SCIENCE 827-834 (1998a); Hegde, R. S., Tremblay, P., Groth, D., DeArmond, S. J., Prusiner, S. B., and Lingappa, V. R. Transmissible and genetic prion diseases share a common pathway of neurodegeneration. 402 NATURE 822-826 (1999)). As ^{Ctm}PrP contains a novel uncleaved, N-terminal signal peptide, mutations in the signal peptide itself also affect the amount of ^{Ctm}PrP. FIG. 3A shows in vitro translation and PK protection assays of wild-type and various mutant PrPs. The full-length forms of ^{Sec}Prp and ^{Ctm}PrP are indicated in FIG. 3 by the white and shaded arrows, respectively (lanes 1, 4, 7, 10). The protease-protected forms of ^{Sec}PrP and ^{Ctm}PrP are indicated by the white and shaded arrows, respectively (lanes 2, 5, 8, 11). FIG. 3B shows FLAG-L9R/3AV PrP synthesized by in vitro translation, and immunoprecipitated with anti-FLAG antibodies M1 (lane 1) or M2 (lane 2). Neither ^{Ctm}PrP (shaded arrowhead) nor untranslocated/unglycosylated PrP (25 kDa) are immunoprecipitated by M1, whereas both forms are immunoprecipitated by M2. FIG. 3C shows BHK cells transiently transfected with plasmids encoding wild-type or mutant PrPs. Post-nuclear supernatants prepared from cells 24 hrs after transfection were incubated with (lanes 2, 3, 5, 6, 8, 9, 11, 12) or without (lanes 1, 4, 7, 10) PK in the presence (lanes 3, 6, 9, 12) or absence (lanes 1, 2, 4, 5, 7, 8, 10, 11) of Triton-X-100 (Det). Proteins were then solubilized in SDS, deglycosylated with PNGase F, and subjected to Western blotting with 3F4 antibody. The protease-protected forms of ^{Sec}Prp and ^{Ctm}PrP are indicated by the white and shaded arrows, respectively.

The substitution of a charged residue for a hydrophobic residue within the signal sequence (L9R) markedly increased the proportion of ^{Ctm}PrP produced after in vitro translation to ˜50% (FIG. 3A, lanes 4 and 5). Combining this mutation with one in the transmembrane domain to create L9R/3AV resulted in a protein that was synthesized exclusively as ^{Ctm}PrP. Before protease treatment, the glycosylated form of this double mutant migrated at 33 kDa (FIG. 3A, lane 7), and after PK digestion an equimolar amount of a 24 kDa protected fragment was produced without any ^{Sec}PrP (lane 8). In experiments where the protected products were inimunoprecipitated and enzymatically deglycosylated prior to SDS-PAGE, a protocol which facilitates detection of ^{Ntm}PrP, no ^{Ntm}PrP was observed. Presence of the uncleaved signal sequence in ^{Ctm}PrP was confirmed by translating a FLAG-tagged version of L9R/3AV, which was not immunoprecipitated with M1 antibody. (FIG. 3B, lane 1).

The L9R mutation altered the topology of PrP in cultured cells as it did after in vitro translation. By carrying out PK protection assays on post-nuclear supernatants prepared from transfected BHK cells, the L9R mutation increased the proportion of ^{Ctm}PrP to about ˜50% (FIG. 3C, lanes 4 and 5), while both untagged and FLAG-tagged versions of L9R/3AV were synthesized entirely as ^{Ctm}PrP (FIG. 3C, lanes 7, 8, 10, 11). (In these procedures, PrP is deglycosylated with PNGase F prior to Western blotting). FLAG-L9R/3AV PrP reacts with P46-66 antibody but not with M1 antibody, indicating that ^{Ctm}PrP has an uncleaved signal peptide when synthesized in BHK cells. Thus, the novel L9R/3AV PrP mutant provides the ability to analyze the properties of ^{Ctm}PrP in a cellular context in the absence of the other two topological variants (^{Sec}PrP and ^{Ntm}PrP).

D. ^{Ctm}PrP Has a GPI Anchor.

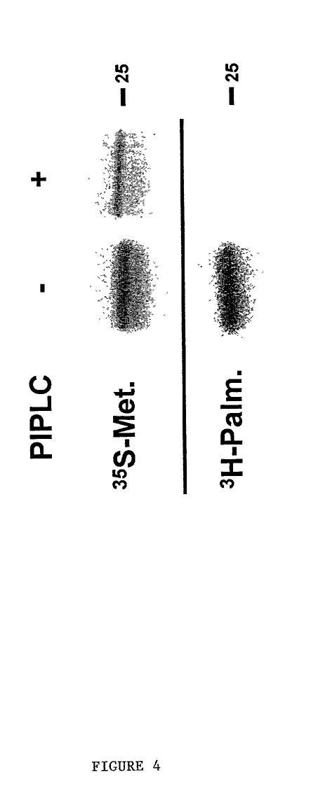

FIG. 4 shows the SDS-PAGE and autoradiography results of tests for the ^{Ctm}PrP GPI anchor. Transfected BHK cells expressing FLAG-L9R/3AV PrP were metabolically labeled for 16 hr with either ³⁵S-methionine (top panel) or ³H-palmitate (bottom panel). Detergent lysates of the cells were deglycoslyated with PNGase F in the presence (+lane) or absence (−lane) of phosphatidylinositol-specific phospholipase C (“PIPLC”) which is known to cleave off the GPI anchor. PrP was then immunoprecipitated with 3F4 antibody and analyzed by SDS-PAGE and autoradiography. The removal of the ³H-palmitate label (lower panel), as well as the slightly reduced mobility of the ³⁵S-methionine-labeled PrP after PIPLC treatment (upper panel) which is characteristic of PrP without its GPI anchor (Narwa, R., and Harris, D. A., Prion Proteins Carrying Pathogenic Mutations are Resistant to Phospholipase Cleavage of their Glycolipid Anchors, 38 BIOCHEM. 8770-8777 (1999), incorporated herein by reference) establishes that ^{Ctm}PrP has such a GPI anchor.

E. ^{Ctm}PrP is Retained in the ER.

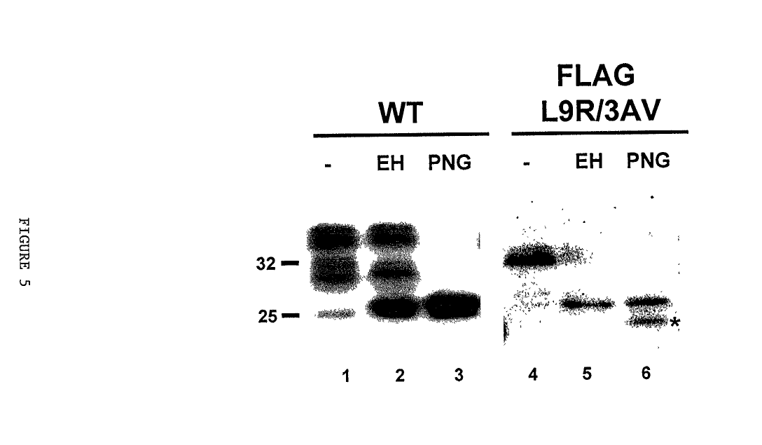

FIG. 5 shows that the oligosaccharide chains of ^{Ctm}PrP are sensitive to digestion with endoglycosidase H (“EH”). Detergent lysates of transfected BHK cells expressing wild-type or FLAG-L9R/3AV PrP were incubated without enzyme (lanes 1 and 4), with endoglycosidase H (lanes 2 and 5) or with PNGase F (lanes 3 and 6). Proteins were then precipitated with methanol and analyzed by Western blotting using antibody 3F4. The band in lane 6 indicated by the asterisk is a proteolytic breakdown product.

FLAG-L9R/3AV PrP synthesized in BHK cells migrated on SDS-PAGE as a sharp band of ˜33 kDa, the same size as core-glycosylated PrP produced by in vitro translation (FIG. 5, lane 4). In contrast, wild-type PrP displayed a sharp band of 25 kDa as well as two broad bands of 28-30 and 33-35 kDa, corresponding to unglycosylated, mono- and di-glycosylated forms (lane 1). This observation suggested the possibility that the oligosaccharide chains of the mutant protein were being only partially processed. Moreover, FLAG-L9R/3AV PrP was quantitatively deglycosylated by endoglycosidase H, which acts only on high-mannose oligosaccharide chains added in the ER (lane 5). In contrast, only a small amount of wild-type PrP was susceptible to this enzyme (lane 2), probably representing molecules in transit to the cell surface. As expected, both proteins were fully deglycosylated by PNGase F, which cleaves both complex and high-mannose oligosaccharide chains (lanes 3 and 6). These results indicate that FLAG-L9R/3AV PrP, and thus ^{Ctm}PrP, does not transit beyond the mid-Golgi stack where oligosaccharides become resistant to endoglycosidase H. Similar results were obtained with an untagged version of L9R/3AV.

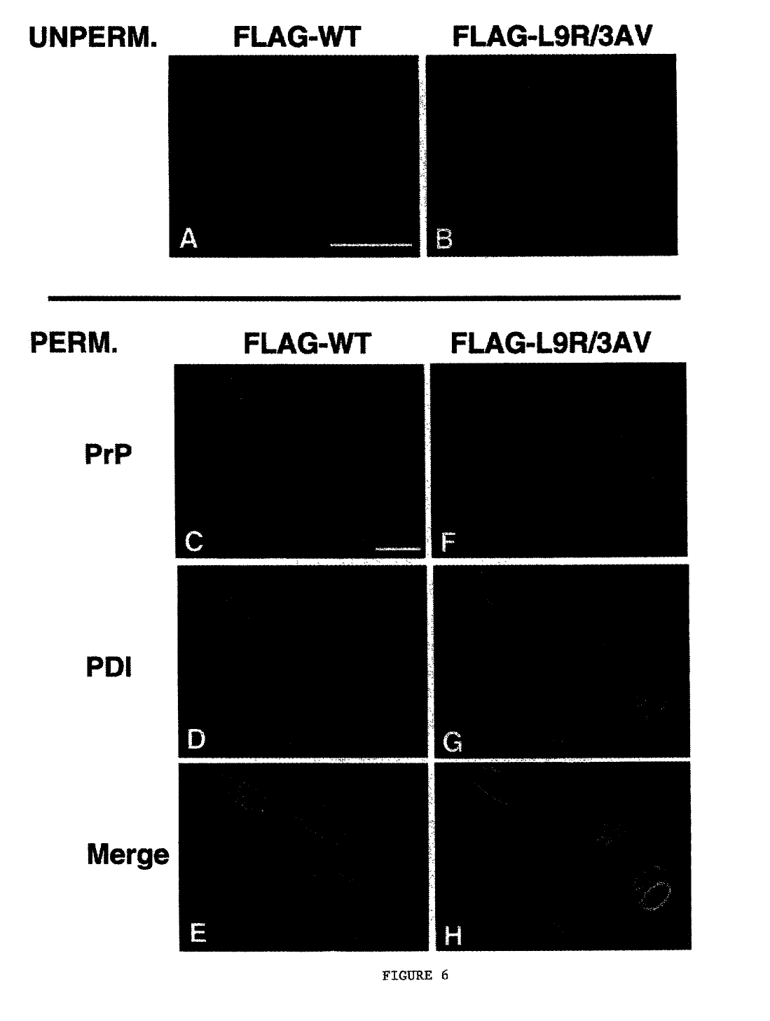

FIG. 6 shows various pictures of immunofluorescence microscopy analysis of ^{Ctm}PrP. FIG. 6 (A, B) shows transfected BHK cells expressing FLAG-wild-type PrP (FIG. 6A) or FLAG-L9R/3AV PrP (FIG. 6B) which were stained with 3F4 antibody without permeabilization in order to visualize PrP on the cell surface. The faint staining visible in panel B represents background, since it is also seen on untransfected cells. In FIG. 6C through H, transfected CHO cells were fixed, permeabilized, and stained with antibodies to PrP (P45-66) and protein disulfide isomerase. After treatment with secondary antibodies, cells were viewed by laser scanning confocal microscopy to visualize PrP (green FIG. 6, panels C and F) or protein disulfide isomerase (red, panels D and G). FIG. 6, panels E and H show a merge of the PrP and protein disulfide isomerase staining patterns. The punctate intracellular staining for PrP seen in panels C and E co-localizes with a Golgi marker. Scale bars in FIG. 6A are 25 micrometers.

In contrast to FLAG-wild-type PrP, FLAG-L9R/3AV PrP was not detectable on the cell surface (FIG. 6A and B). When cells were detergent-permeabilized, FLAG-L9R/3AV PrP was distributed within the cell in a reticular pattern that largely colocalized with the ER marker protein disulfide isomerase (FIG. 6F-H), while FLAG-wild-type PrP was largely localized to the cell surface, with a small amount of internal punctate staining that corresponded to the Golgi (FIG. 6C-E). Results similar to those shown in FIG. 6 were obtained when non-FLAG versions of wild-type and L9R/3AV PrP were used. Moreover, wild-type PrP but not L9R/3AV PrP could be labeled on intact cells using a membrane-impermeant biotinylation reagent, further confirming the absence of the mutant protein from the cell surface.

F. Establishment of L9R/3AV Expressing Transgenic Mice.

Transgenic mice expressing 3AV PrP develop scrapie-like neurological disease without PrP^{Sc}. (Hedge, R. S., Mastrianni, J. A., Scott, M. R., Defea, K. A., Tremblay, P., Torchia, M., DeArmond, S. J., Prusiner, S. B., and Lingappa, V. R., A transmembrane From of the Prion Protein in Neurodegenerative Disease, 279 SCIENCE 827-834 (1998a). The authors estimated the fraction of ^{Ctm}PrP present in the brains of these mice at 10-20%. In accordance with the invention, novel transgenic mice have been created that express L9R/3AV mouse PrP under control of either a modified PrP promoter (referred to as Tg(L9R/3AV^{PrP}) mice), or a tetracycline-regulated promoter (referred to as Tg(L9R/3AV^{tetO}) mice). Since the mutant protein will be expressed exclusively in the ^{Ctm}PrP orientation, these mice are likely to develop a severe neurological illness. The procedures for construction of the mice are given below, and some of them are also described in Chiesa, R., Piccardo, P., Ghetti, B., and D. A. Harris, Neurological Illness in Transgenic Mice Expressing a Prion Protein with an Insertional Mutation, 21 NEURON, 1339-1351 (1998).

For construction of novel Tg(L9R/3AV^{PrP}) mice, the open reading frame of L9R/3AV mouse PrP was amplified by PCR using KlenTaq LA polymerase (Sigma) with sense (GACCAGCTCGAGATGGCGAACCTTGGCTACTGG) and anti-sense (GACCAGCTCGAGTCATCCCACGATCAGGAAGAT) primers containing XhoI sites. PCR products were digested with XhoI, and ligated into the expression vector MoPrP.Xho (Borchelt, D. R., Davis, J., Fischer, M., Lee, M. K., Slunt, H. H., Ratovitsky, T., Regard, J., Copeland, N. G., Jenkins, N. A., Sisodia, S. S., and Price, D. L. (1996). A Vector For Expressing Foreign Genes In The Brains And Hearts Of Transgenic Mice, GENET. ANAL. BIOMOL. ENG. 13, 159-163) that had also been cut with XhoI. Recombinant plasmids with inserts in the correct orientation were selected by PCR screening of bacterial colonies using primers P1 and P4 (see below), and their identity was confirmed by restriction analysis and sequencing of the coding regions in their entirety. Transgenes were excised from the recombinant plasmids by digestion with NotI, purified on NACS-52 Prepac columns (Gibco BRL) and injected into the pronuclei of fertilized eggs from an F₂ cross of C57BL/6J x CBA/J F₁ parental mice. Transgenic founders were bred to C57BL/6J x CBA/J F₁ parental mice.

For construction of Tg(L9R/3AV^{tetO}) mice, the transgenic construct used to create the Tg(L9R/3AV^{PrP}) mice was digested with XbaI, and a 6.6 kb fragment was isolated and subcloned into the XbaI site of pBluescript. The resulting plasmid was then digested with NotI and SalI, and the insert cloned into the vector pBI-G (Clontech) that had been digested with the same enzymes. The transgene was then isolated from the resulting plasmid by restriction digestion, and was purified and injected into fertilized eggs as described above.

The PrP gene status of weanling mice was determined by PCR analysis of tail DNA prepared with a Puregene DNA Isolation Kit from Gentra Systems. Primers P1 and P4 were used to detect the presence of the transgene, since they amplify an 842 bp segment of the transgene, but they do not amplify the endogenous Prn-p gene. PCR was performed at 94° C. (30 sec), 50° C. (30 sec), and 72° C. (45 sec) for 40 cycles. Primer sequences were: CTTCAGCCTAAATACTGG (P1); and CACGAGAATGCGAAGG (P4).

Among other applications known to those skilled in the art, the inventive Tg(L9R/3AV^{PrP}) and Tg(L9R/3AV^{tetO}) mice may be used to: (1) confirm that ^{Ctm}PrP is a pathogenic molecule; (2) test hypotheses about how ^{Ctm}PrP causes neurodegeneration; and (3) provide a standardized source of ^{Ctm}PrP for the calibration of diagnostic assays and for further characterization of this form of PrP.

G. Development of Diagnostic Assays for ^{Ctm}PrP.

Detection of ^{Ctm}PrP is difficult with existing methods, even in transgenic animals, due to the presence of much larger amounts of the other topological forms of the protein (^{Sec}Prp and ^{Ntm}Prp). The protease protection assay currently used is cumbersome, and impossible to perform on post-mortem samples, making its diagnostic use unlikely. The presence of an uncleaved signal peptide in ^{Ctm}PrP provided by the present invention, establishes means of selectively recognizing this form of PrP, even in the presence of the other topological variants. Several techniques can be used to detect ^{Ctm}PrP by virtue of the novel retained signal peptide including, without limitation, the following examples. First, monoclonal and polyclonal antibodies raised against the signal peptide itself (residues 1-22), or against peptides spanning the signal peptide cleavage site (at codons 22/23) can be used in Western blot, immunoprecipitation, or ELISA formats to measure ^{Ctm}PrP. Second, a cysteine residue is present at amino acid 22, just before the signal cleavage site which can be used as a target for labeling with sulfhydryl-reactive reagents. These reagents can be either radioactive or biotinylated, and can be used to recognize ^{Ctm}PrP in Western blot, immunoprecipitaton, or ELISA formats. Third, the presence of the cysteine residue in the signal sequence of PrP means that ^{Ctm}PrP can form disulfide-linked dimers migrating at ˜65 kDa on non-reducing SDS-PAGE, providing a means by which to selectively recognize this molecule based on its molecular size on Western blots. Since the cysteine residue in question is absolutely conserved in mammalian PrPs, methods involving the reactivity of this cysteine may be used as a pre or post mortem diagnostic tool for the presence of ^{Ctm}PrP in human as well as animal tissues.

H. Development of Novel Molecular Markers for Prion Diseases.

This invention provides clues to the mechanisms by which ^{Ctm}PrP might play a role in the pathogenesis of prion diseases and suggests novel molecular markers that can be used for the diagnosis of these diseases, and for following their clinical course. Hegde et al. have hypothesized that ^{Ctm}PrP is a component of a common pathway of neurodegeneration underlying both infectious and genetic forms of prion diseases, and that PrP^{Sc} is pathogenic because it enhances the formation of ^{Ctm}PrP (Hegde, R. S., Tremblay, P., Groth, D., DeArmond, S. J., Prusiner, S. B., and Lingappa, V. R., Transmissible and Genetic Prion Diseases Share a Common Pathway of Neurodegeneration. 402 NATURE 822-826 (1999)). This hypothesis is based on indirect evidence that levels of ^{Ctm}PrP in mouse brain increase during the course of scrapie infection, and on the finding that transgenic mice expressing PrP with ^{Ctm}PrP-favoring mutations develop a scrapie-like neurological illness without PrP^{Sc}. The inventive discoveries described herein, including that ^{Ctm}PrP is retained in the ER, suggests that ^{Ctm}PrP may damage neurons by activating stress-induced signaling pathways that are engaged by the accumulation of misfolded proteins in the ER. Some of these pathways, such as the unfolded protein response that results in up-regulation of ER chaperone synthesis, are adaptive in nature, but others, such as induction of the transcription factor CHOP/GADD153 and phosphorylation of the translation initiation factor eIF-2, can kill cells by an apoptotic mechanism (Chapman, R., Sidrauski, C., and Walter, P., Intracellular Signaling from the Endoplasmic Reticulum to the Nucleus, 14 ANNU. REV. CELL DEV. BIOL. 459-485 (1998); Kaufman, R. J., Stress Signaling from the Lumen of the Endoplasmic Reticulum: Coordination of Gene Transcriptional and Translational Controls, 13 GENES DEV. 1211-1233 (1999), both incorporated herein by reference). Thus, the present invention links prion diseases for the first time to other inherited, human disorders that are due to retention of misfolded proteins in the ER (Perlmutter, D. H., Misfolded Proteins in the Endoplasmic Reticulum, 79 LAB. INVEST. 623-638 (1999), incorporated herein by reference). Thus, components of ER stress response pathways may be used as molecular markers for the diagnosis of prion diseases, and for monitoring their clinical course. Such components include, but are not limited to, Binding Protein (BiP), glucose-regulated protein 94 (Grp94), protein disulfide isomerase (PDI), transcription factor CHOP/GADD153, and phosphorylated elongation initiation factor 2 (eIF-2). The amounts of these components may be assayed Western blot, ELISA or other means in tissue samples or biological fluids.

It is to be understood that although the description herein of the invention is only illustrative, none of the embodiments or examples shown herein are limiting. It is apparent to those skilled in the art that modifications and adaptation can be made without departing from the scope of the invention.

We claim:

1. A method for selective recognition of ^{Ctm}PrP in a mammal comprising identifying the presence of an uncleaved signal peptide on said ^{Ctm}PrP.

2. The method of claim 1 wherein said signal peptide comprises the first 22 amino acids of SEQ ID: 5.

3. The method of claim 1 wherein antibodies that bind specifically to the uncleaved signal peptide region on ^{Ctm}PrP or an immunogenic fragment thereof are used to identify the ^{Ctm}PrP.

4. The method of claim 1 where antibodies that bind to the first 22 amino acids of SEQ ID: 5 or an immunogenic fragment thereof are used to identify the ^{Ctm}PrP.

5. The method of claim 1 where antibodies that bind specifically to the region of the cleavage site of the uncleaved signal peptide on ^{Ctm}PrP are used to identify the ^{Ctm}PrP.

6. The method of claim 1 wherein a cysteine amino acid, present within the signal peptide, is targeted with sulfhydryl-reactive reagents to identify the ^{Ctm}PrP.

7. The method of claim 6 wherein said cysteine amino acid is present at amino acid 22 of the signal peptide.

8. The method of claim 1 wherein a cysteine amino acid, present within the signal peptide, is used to form disulfide-linked dimers to identify the ^{Ctm}PrP.

9. The method of claim 8 where said cysteine amino acid is present at amino acid 22 of the signal peptide.

10. An antibody that binds specifically to the uncleaved signal peptide region on ^{Ctm}PrP or an immunogenic fragment of said signal peptide region.

11. An antibody that binds specifically to the first 22 amino acids of SEQ ID: 5.

12. An antibody that binds specifically to the region of the cleavage site of the uncleaved signal peptide on ^{Ctm}PrP.

13. A method of diagnosing prion caused disease in a mammalian patient by identifying the presence of an uncleaved signal peptide on ^{Ctm}PrP.

14. The method of claim 13 wherein the patient is a human.

15. The method of claim 13 wherein the patient is a livestock animal.

16. The method of claim 13 wherein the identification is performed post-mortem.

17. A method of diagnosing prion caused diseases in a mammalian patient comprising utilizing components of endoplasmic reticulum stress response pathways as molecular markers.

18. An isolated nucleic acid comprising a sequence that encodes a polypeptide having the sequence of SEQ ID: 8 and conservative amino acid substitutions therein.

19. An isolated nucleic acid comprising the nucleic acid sequence of SEQ ID: 4 or a degenerate variant of SEQ ID: 4.

20. An isolated nucleic acid comprising a sequence that encodes a polypeptide having the sequence of SEQ ID: 6 and conservative amino acid substitutions therein.

21. An isolated nucleic acid comprising the nucleic acid sequence of SEQ ID: 2 or a degenerate variant of SEQ ID: 2.

22. An expression vector comprising the nucleic acid of SEQ ID: 4 operably linked to an expression control sequence.

23. An expression vector comprising the nucleic acid of SEQ ID: 2 operably linked to an expression control sequence.

24. A cultured cell comprising the vector of claim 22.

25. A cultured cell comprising the nucleic acid of claim 18.

26. A cultured cell comprising the nucleic acid of claim 19.

27. A cultured cell comprising the vector of claim 23.

28. A cultured cell comprising the nucleic acid of claim 20.

29. A cultured cell comprising the nucleic acid of claim 21.

30. A method of expressing ^{Ctm}PrP in a cell comprising:
providing an expression vector having SEQ ID: 4 or degerate variants thereof, introducing the vector into a cell; and
maintaining the cell under conditions permitting expression of ^{Ctm}PrP in the cell.

31. A method of expressing ^{Ctm}PrP in a cell comprising:
providing an expression vector having SEQ ID: 2 or degerate variants thereof, introducing the vector into a cell; and
maintaining the cell under conditions permitting expression of ^{Ctm}PrP in the cell.

32. Isolated ^{Ctm}PrP produced by the method of claim 30.

33. Isolated ^{Ctm}PrP produced by the method of claim 31.

34. A transgenic, non-human animal comprising the vector of claim 22.

35. A transgenic, non-human animal comprising the vector of claim 23.

36. A transgenic mouse comprising the vector of claim 22.

37. A transgenic mouse comprising the vector of claim 23.

38. Isolated ^{Ctm}PrP produced by the animal of claim 34.

39. Isolated ^{Ctm}PrP produced by the animal of claim 35.

40. Isolated ^{Ctm}PrP produced by the mouse of claim 36.

41. Isolated ^{Ctm}PrP produced by the mouse of claim 37.
